(19) United States
(12) Patent Application Publication
(10) Pub. No.: US 2005/0272923 A1
(43) Pub. Date: Dec. 8, 2005

(54) Mature microRNA prediction method using bidirectional hidden markov model and medium recording computer program to implement the same

(76) Inventors: Byoung-Tak Zhang, Seoul (KR); Jin-Wu Nam, Anyang-Si (KR); Ki-Roo Shin, Seoul (KR)

Correspondence Address: HARNESS, DICKEY & PIERCE, P.L.C., P.O. BOX 828, BLOOMFIELD HILLS, MI, 48303, US

(21) Appl. No.: 11/121,168

(22) Filed: May 3, 2005

(30) Foreign Application Priority Data
May 6, 2004 (KR) 10-2004-0032005

(51) Int. Cl. C07H 19/00

(52) U.S. Cl. 536/22.1

(57) ABSTRACT

Disclosed are a method of predicting mature microRNA regions using a bidirectional hidden Markov model and a medium recording a computer program to implement the method. The method includes representing each base pair comprising the microRNA precursor by state information of match, mismatch and bulge states; representing the base pair by a basepair emission symbol; computing a Viterbi probability (P) for microRNA using a probability (Eₛ(q)) that state s emits symbol q and a transition probability (T_{ab}) from state a to state b; computing a Viterbi probability (Pₜ(i)) that the i-th base pair is true and another Viterbi probability (P_{f}(i)) that the i-th base pair is false; and computing a position probability (S(i)) for mature microRNA using the Viterbi probability, wherein, if the position probability (S(i)) for mature microRNA is greater than a predetermined value, the position at which the base pair is present is taken as the mature microRNA region. The method of predicting a mature microRNA region makes it possible to perform learning and searching for a shorter period of time and has high prediction efficiency. Also, the method is capable of identifying microRNA genes and predicting mature microRNA regions at the same time. Thus, the present invention has a beneficial effect of supplying a much larger amount of information.

### BACKGROUND OF THE INVENTION

1. Field of the Invention

The present invention relates to a method of predicting mature microRNA regions using a bidirectional hidden Markov model and a medium on which a computer program is recorded to implement the method. More particularly, the present invention relates to a method of predicting mature microRNA regions using a bidirectional hidden Markov model, which is based on learning structure information and sequence information at the same time using a hidden Markov model, which is a probabilistic model, to identify structurally similar microRNA genes in the human genome, and identifying microRNA genes, which are a class of small non-coding RNAs, using the learned model, and a medium on which a computer program is recorded to implement the method.

2. Description of the Prior Art

MicroRNA (also called miRNA) is a sort of small RNA, and has been newly identified to directly regulate gene expression by arresting mRNA translation. Thus, identification of microRNA in the genome database is very important in biology. In humans, more than 150 microRNAs have been identified so far, but a large number of human microRNAs remains unidentified.

One important problem in the identification of microRNA is to accurately predict actual mature microRNA regions over microRNA precursors. A microRNA precursor of about 70 nucleotides (nt) in length is processed to a mature microRNA of about 22 nt by an enzyme protein called “Dicer”. Another problem involves the prediction of a cleavage site recognized by Dicer in a microRNA precursor.

Some computational approaches were conventionally introduced to predict microRNA genes. One approach involves analyzing statistical data of microRNA genes from related species to identify homologous microRNA precursors. Although this approach provides significant results, it is problematic in terms of being unable to find putative microRNA precursors when microRNA precursors of related species are not known and statistical data are thus not established.

The second approach, which is similar to the first approach, is based on finding common hairpin structures shared by mosquitoes and Drosophila species and finding sequences similar to microRNA found in drosophilae from the common hairpin structures. However, this algorithm does not give significant results due to its very low efficiency.

The third approach is to predict microRNA using a genetic programming technique that automatically learns common structures of microRNAs from a set of known microRNA precursors. This algorithm has good performance, but has the disadvantage of requiring a lot of time to learn.

### SUMMARY OF THE INVENTION

Accordingly, the present invention has been made keeping in mind the problems occurring in the prior art, and an object of the present invention is to provide a method of predicting a mature microRNA region using a bidirectional hidden Markov model, which is based on identifying microRNA in the genome database using a probabilistic model, thereby greatly reducing the time and expense required for biological experiments and providing an easy approach.

Another object of the present invention is to provide a medium on which a computer program is recorded to implement the method.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a representation showing a stem-loop secondary structure of a microRNA precursor and match states and symbols of a hidden Markov model;

FIG. 2 is a transition diagram constructed for a bidirectional hidden Markov model;

FIG. 3 is a graph showing the prediction performance of the mature microRNA region prediction method according to an embodiment of the present invention;

FIG. 4 shows the secondary structures of the predicted microRNA gene candidates on human chromosome 19 and mouse microRNA genes; and

FIG. 5 is a graph showing the signal S(i) of a human microRNA gene has-let-7a-3.

### DETAILED DESCRIPTION OF THE INVENTION

The present invention, which has been made to solve the problems encountered in the prior art, is directed to a method of predicting a mature microRNA region contained in a microRNA precursor. The method comprises representing each base pair comprising the microRNA precursor by state information of match, mismatch and bulge states; representing the base pair by a basepair emission symbol; computing a Viterbi probability (P) for microRNA using a probability (Eₛ(q)) that state s emits symbol q and a transition probability (T_{ab}) from state a to state b according to the following equation;

$$P = {E}_{s ( q1 )} ( {q}_{1} ) \cdot {\prod }_{i = 2}^{22} \{ {T}_{s ( {q}_{i - 1} ) s ( {q}_{i} )} \cdot {E}_{s ( {q}_{i} )} ( {q}_{i} ) \}$$

computing a Viterbi probability (Pₜ(i)) that the i-th base pair is true and another Viterbi probability (P_{f}(i)) that the i-th base pair is false according to the following equations; and
P_{τ}(i)=max{P_{τ}(i−1)·T_{τ(q}_{ᵢ₋₁}_{)τ(q}_{ᵢ}₎, P_{f}(i−1)·T_{υ(q}_{ᵢ₋₁}_{)τ(q}_{ᵢ}₎}·E_{τ(q}_{ᵢ}₎(qᵢ)
P_{f}(i)=max{P_{τ(q}_{ᵢ₋₁}_{)υ(q}_{ᵢ}₎, P_{f}(i−1)·T_{υ(q}_{ᵢ₋₁}_{)υ(q}_{ᵢ}₎}·E_{υ(q}_{ᵢ}₎(qᵢ)

computing a position probability (S(i)) for mature microRNA using the Viterbi probability according to the following equation,

$$S ( i ) = \frac{{P}_{t} ( i - 1 ) \cdot {T}_{τυ}}{{P}_{t} ( i - 1 ) \cdot {T}_{τυ} + {P}_{f} ( i - 1 ) {T}_{υυ}}$$

wherein, if the position probability (S(i)) for mature microRNA is greater than a predetermined value, the position at which the base pair is present is determined as the mature microRNA region.

The match state (M) is represented by any emission symbol among A-U, U-A, G-C, C-G, U-G and G-U. The bulge state (B) is represented by any emission symbol among A-, U-, G-, C-, -A, -U, -G and -C. The mismatch state (N) is represented by any one of the remaining emission symbols.

A position probability for mature microRNA, in a direction from the stem to the loop of the microRNA precursor, and another position probability for mature microRNA, in a direction from the loop to the stem of the microRNA precursor, are computed. The position of a base pair, at which the values of the position probabilities form peaks, is taken as an end point of the mature microRNA region.

In addition, the present invention includes a medium on which a computer program is recorded to implement the method of predicting a mature microRNA region using a bidirectional hidden Markov model.

Hereinafter, the present invention will be described with reference to the accompanying drawings. The following embodiment is set forth to illustrate, but is not to be construed as the limit of the present invention.

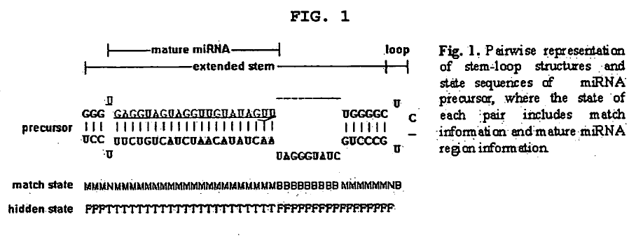

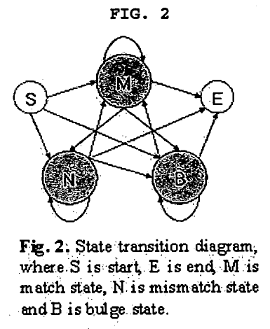

FIG. 1 is a representation showing the stem-loop secondary structure of a microRNA precursor and match states and symbols of a hidden Markov model. FIG. 2 is a transition diagram constructed for a bidirectional hidden Markov model.

Since the statistical information is insufficient for primary nucleotide sequences of microRNA genes, it is difficult to identify microRNA genes and predict mature microRNA regions using conventional computational algorithms. In this regard, based on the fact that microRNAs have higher similarity in secondary structures than in nucleotide sequences, the present inventors developed a method of simultaneously expressing sequence information and secondary structure information as a probability model. A microRNA precursor can be represented by a secondary structure in which each base pair is present in a match, mismatch or bulge state. Each symbol to be emitted is a base pair. The hidden Markov model learns bidirectionally, that is, both in a forward direction from the stem to the loop of the microRNA precursor and in a backward direction from the loop to the stem of the microRNA precursor, and uses each model at the same time for prediction.

This research is gaining much interest worldwide, and many researchers have made efforts to develop microRNA prediction algorithms. However, a general algorithm has not been developed yet. The present invention relates to an algorithm that is the first to have the features of a general algorithm applicable to humans and other species, and was made using a bidirectional hidden Markov model developed by the present inventors.

Referring to FIG. 1, a microRNA precursor has a stem-loop structure and may be expressed as a hidden Markov model using information at each position of the stem-loop structure. First, the microRNA precursor may be represented by state information of match, mismatch or bulge states. Second, each state may be represented by emission information. The match state (M) emits any symbol among A-U, U-A, G-C, C-G, U-G and G-U. The bulge state (B) emits any symbol among A-, U-, G-, C-, -A, -U, -G and -C. The mismatch state (N) emits any one of the remaining the basepair symbols. The possible transitions among the three match states are shown in FIG. 2.

A hidden Markov model is learned from previously known nucleotide sequences of human microRNA precursors. The state of each microRNA in the genome and optimized paths of emission symbols are searched for through the variation of the Viterbi algorithm. In the present invention, the Viterbi probability (P) for microRNA is computed according to an Equation 1, below. When the P value is greater than a predetermined value, a given candidate is classified as a microRNA gene.

$$P = {E}_{s ( q1 )} ( {q}_{1} ) \cdot {\prod }_{i = 2}^{22} \{ {T}_{s ( {q}_{i - 1} ) s ( {q}_{i} )} \cdot {E}_{s ( {q}_{i} )} ( {q}_{i} ) \} \qquad [ Equation 1 ]$$

wherein, Eₛ(q) is the probability that state s emits symbol q, and (T_{ab}) is the transition probability from state a to state b. Thus, T_{s(q}_{ᵢ₋₁}_{)s(q}_{ᵢ}₎ means the transition probability from the i−1-th state of symbol qᵢ₋₁ to the i-th state of symbol qᵢ. In the present invention, the probability for microRNA of about 21 base pairs in length is computed.

In addition, in order to predict a mature microRNA region in the microRNA precursor, a Viterbi probability (Pₜ(i)) that the i-th position is true and another Viterbi probability (P_{f}(i)) that the i-th position is false are computed according to Equations 2 and 3, below.
P_{τ}(i)=max{P_{τ}(i−1)·T_{τ(q}_{ᵢ₋₁}_{)τ(q}_{ᵢ}₎, P_{f}(i−1)·T_{υ(q}_{ᵢ₋₁}_{)τ(q}_{ᵢ}₎}·E_{τ(q}_{ᵢ}₎(qᵢ) [Equation 2]
P_{f}(i)=max{P_{τ(q}_{ᵢ₋₁}_{)υ(q}_{ᵢ}₎, P_{f}(i−1)·T_{υ(q}_{ᵢ₋₁}_{)υ(q}_{ᵢ}₎}·E_{υ(q}_{ᵢ}₎(qᵢ) [Equation 3]

wherein, τ(q) is the true state of symbol q, υ(q) is the false state of symbol q, and the initial condition is Pₜ(1)=0, P_{f}(1)=1.

However, it is difficult to accurately predict mature microRNA regions using only the Viterbi probabilities. Thus, a position probability (S(i)) for mature microRNA is computed from a value calculated using the probability of the transition to false states, according to Equation 4, below, and a mature microRNA region is finally determined. When the S(i) value is greater than a predetermined value, a given position is predicted as a mature microRNA region.

$$S ( i ) = \frac{{P}_{t} ( i - 1 ) \cdot {T}_{τ υ}}{{P}_{t} ( i - 1 ) \cdot {T}_{τυ} + {P}_{f} ( i - 1 ) {T}_{υυ}} \qquad [ Equation 4 ]$$

The equations given above give a signal in a direction from the stem to the loop of the microRNA precursor, that is, a forward signal. Thus, the hidden Markov model is learned backwards, that is, in a direction from the loop to the stem, and the aforementioned computation is repeated. In the backward processing, the i index of each base pair is reversely represented.

### Test Results

A microRNA prediction test in the present invention included evaluating the performance of the present algorithm and predicting microRNA genes on human chromosomes 18 and 19.

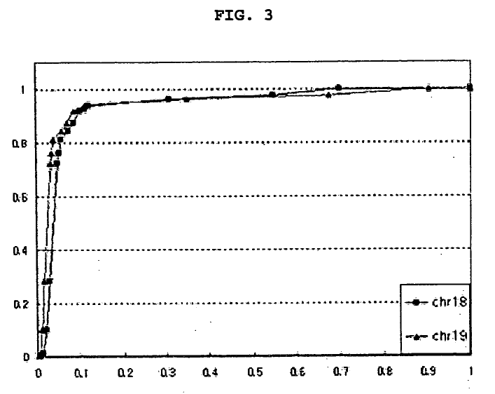

FIG. 3 is a graph showing the prediction performance of the mature microRNA prediction method according to an embodiment of the present invention. FIG. 3 shows the results of 5-fold cross-validation of 136 known human microRNAs that were randomly divided into five subsets. The prediction method according to the embodiment of the present invention displayed 72.8% sensitivity and 95.9% specificity on average. These results indicate that the present method provides more reliable results than conventional methods.

TABLE 1

| | Size of chr | Stem- | Precursor | | Expression | Known | Detected | Homolo | Contained | |
|---|---|---|---|---|---|---|---|---|---|---|
| Chr | (Mop) | loop | Candidates | Percentage (%) | Verified | mRNA | mRNA | [symbol] | partial [symbol] | Intron |
| 18 | 56.7 | 34853 | 2253 | 6.46 | 84 | 2 | 2 | 22 | 8 | 0 |
| 19 | 75.7 | 62229 | 2065 | 3.32 | 171 | 5 | 4 | 42 | 12 | 3 |

Table 1, above, shows the microRNA prediction results of chromosomes 18 and 19. The predicted microRNA precursors were subjected to human EST (Expressed Sequence Taq) analysis to determine whether they are actually expressed in cells. 2253 and 2065 microRNA precursor candidates on chromosomes 18 and 19, respectively, were found. 84 of 2253 candidates and 171 of 2065 candidates were found in the human EST database, indicating that they are actually transcribed in cells. Also, the candidates were found to include six of seven previously known microRNAs on chromosomes 17 and 18.

TABLE 2

| | Criterion | | | | | | | |
|---|---|---|---|---|---|---|---|---|
| | Mean of | | | | Square root of the | | | |
| | absolute distance | | | | mean of the squares | | | |
| | 5′ sense | | 3′ anti-sense | | 5′ sense | | 3′ anti-sense | |
| | start | end | Start | and | start | End | start | end |
| Total | 2.83 | 3.31 | 2.42 | 2.15 | 4.16 | 5.11 | 3.32 | 3.65 |
| Total except | 1.96 | 2.47 | 2.13 | 1.60 | 2.56 | 3.26 | 2.70 | 2.14 |
| failures | | | | | | | | |
| (68 + 48) | | | | | | | | |

Table 2, above, shows the error rates of mature microRNA region prediction using a total of 116 known microRNA precursor data. Mature microRNA is located in either a 5′-sense strand or a 3′-antisense strand. Errors at start and end regions of each strand are shown in Table 2. Except for prediction failures, the variation of the mature miRNA region prediction results was an average of 1.96 nucleotides at the start region and an average of 2.47 nucleotides at the end region for 5′-sense strand microRNA genes. For 3′-antisense strands, the variation was 2.13 nucleotides at the start region and 1.60 nucleotides at the end region. These results indicate that the present algorithm gives better prediction results for 3′-antisense strands.

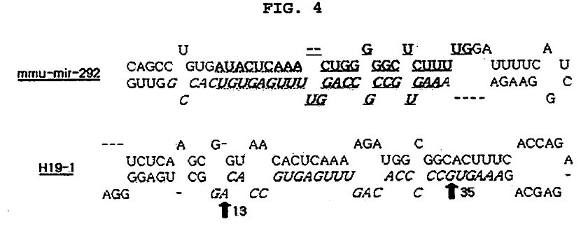

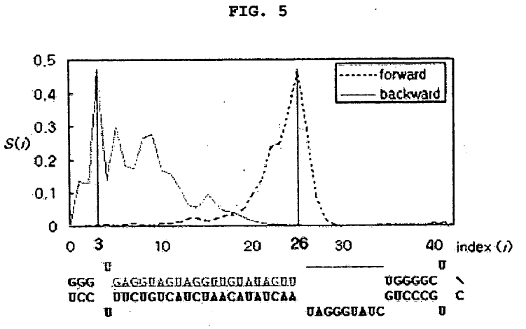

FIG. 4 shows the secondary structures of the predicted microRNA gene candidates on human chromosome 19 and mouse microRNA genes. FIG. 5 is a graph showing the signal S(i) of a human microRNA gene, hsa-let-7a-3.

When the most likely microRNA candidate was analyzed, the mature microRNA region of the putative microRNA was found to be almost identical to that of mice. Also, the position probability, that is, the signal S(i), for mature microRNA in the putative microRNA was observed, and FIG. 5 shows the signal of previously known hsa-let-7a-3.

Although a preferred embodiment of the present invention has been described for illustrative purposes, the embodiment is set forth to illustrate but is not to be construed as the limit of the present invention, and those skilled in the art will appreciate that various modifications, additions and substitutions are possible, without departing from the scope and spirit of the invention as disclosed in the accompanying claims.

The present invention has been implemented using the C++ language and constructed in the form of being executable over the web, but may also be implemented through other languages.

As described hereinbefore, the present invention provides a method of predicting a mature microRNA region, which performs learning and searching for a shorter period of time and has high prediction efficiency. Also, the present invention makes it possible to identify microRNA genes and predict mature microRNA regions at the same time. Thus, the present invention has a beneficial effect of supplying a much larger amount of information.

1. A method of predicting a mature microRNA region contained in a microRNA precursor, comprising:
representing each base pair comprising the microRNA precursor by state information of match, mismatch and bulge states;
representing the base pair by a basepair emission symbol;
computing a Viterbi probability (P) for microRNA using a probability (Eₛ(q)) that state s emits symbol q and a transition probability (T_{ab}) from state a to state b according to the following equation;
$$P = {E}_{s ( q1 )} ( {q}_{1} ) \cdot {\prod }_{i = 2}^{22} \{ {T}_{s ( {q}_{i - 1} ) s ( {q}_{i} )} \cdot {E}_{s ( {q}_{i} )} ( {q}_{i} ) \}$$
computing a Viterbi probability (Pₜ(i)) that the i-th base pair is true and another Viterbi probability (P_{f}(i)) that the i-th base pair is false according to the following equations; and
P_{τ}(i)=max{P_{τ}(i−1)·T_{τ(q}_{ᵢ₋₁}_{)τ(q}_{ᵢ}₎, P_{f}(i−1)·T_{υ(q}_{ᵢ₋₁}_{)τ(q}_{ᵢ}₎}·E_{τ(q}_{ᵢ}₎(qᵢ)
P_{f}(i)=max{P_{τ(q}_{ᵢ₋₁}_{)υ(q}_{ᵢ}₎, P_{f}(i−1)·T_{υ(q}_{ᵢ₋₁}_{)υ(q}_{ᵢ}₎}·E_{υ(q}_{ᵢ}₎(qᵢ)
computing a position probability (S(i)) for the mature microRNA region using the Viterbi probability according to the following equation,
$$S ( i ) = \frac{{P}_{t} ( i - 1 ) \cdot {T}_{τυ}}{{P}_{t} ( i - 1 ) \cdot {T}_{τ υ} + {P}_{f} ( i - 1 ) {T}_{υυ}}$$
wherein, if the position probability (S(i)) for mature microRNA is greater than a predetermined value, the position at which the base pair is present is taken as the mature microRNA region.

2. The method of predicting the mature microRNA region as set forth in claim 1, wherein the match state is represented by any emission symbol among A-U, U-A, G-C, C-G, U-G and G-U, the bulge state is represented by any emission symbol among A-, U-, G-, C-, -A, -U, -G and -C, and the mismatch state is represented by any one of remaining emission symbols.

3. The method of predicting the mature microRNA region as set forth in claim 2, wherein a position probability for mature microRNA in a direction from stem to loop of the microRNA precursor and another position probability for mature microRNA in a direction from loop to stem of the microRNA precursor are computed, and the position of a base pair, at which the values of the position probabilities form peaks, is determined as an end point of the mature microRNA region.

4. A medium on which a computer program is recorded to implement the method of predicting the mature microRNA region using the bidirectional hidden Markov model according to any one of claims 1 to 3.
